(19) United States
(12) Patent Application Publication
(10) Pub. No.: US 2009/0136948 A1
(43) Pub. Date: May 28, 2009

(54) NANOCONFINEMENT- BASED DEVICES AND METHODS OF USE THEREOF

(76) Inventors: Jongyoon HAN, Bedford, MA (US); Reto B. SCHOCH, Speicherchwendi (CH); Lih Feng CHEOW, Cambridge, MA (US)

Correspondence Address: Pearl Cohen Zedek Latzer, LLP, 1500 Broadway, 12th Floor, New York, NY, 10036, US

(21) Appl. No.: 12/261,546

(22) Filed: Oct. 30, 2008

Related U.S. Application Data
(60) Provisional application No. 61/001,105, filed on Oct. 31, 2007.

(51) Int. Cl. C12Q 1/68; B01J 19/00; C12M 1/00; G01N 21/64; G01N 27/00; C12Q 1/02; G01N 33/00; G01N 33/53

(52) U.S. Cl. 435/6; 422/68.1; 435/287.1; 422/82.08; 422/82.02; 435/29; 436/94; 436/86; 435/7.8

(57) ABSTRACT

The present invention provides a device/kit and methods of use thereof in rapid detection of target molecule binding to a cognate binding partner. The methods, inter-alia, make use of a device comprising channels or reservoirs, which are linked to nanochannels, whereby upon application of the cognate binding partner to the nanochannel comprising the target molecule under flow, a detectable change in conductance, capacitance or fluorescence or surface potential occurs.

### CROSS-REFERENCE TO RELATED APPLICATION

This application claims priority under 35 U.S.C. § 119(e) to U.S. provisional patent application Ser. No. 61/001,105, filed Oct. 31, 2007, and is incorporated herein by reference in its entirety.

### GOVERNMENT SUPPORT

This invention was made in whole or in part with U.S. Government support from the National Institute of Health, Grant Number NIH EB005743. The government has certain rights in the invention.

### FIELD OF THE INVENTION

This invention provides devices and methods for rapid analyte detection.

### BACKGROUND OF THE INVENTION

Lab-on-chip devices and applications represent a cost-effective means for rapid throughput assay and detection of materials of interest. The devices are particularly desirable for detection and assay of low-abundance samples, yet such devices suffer a number of limitations to date. Analyte detection, for example, by immunoassay, in such devices is limited, inter alia, by the existence of surface diffusion layers in such devices, which limits the binding kinetics. In typical ELISA or bead-based immunoassays, target molecules need to be transported (primarily by diffusion) to the surface-bound antibodies for a binding reaction to occur. The distance for this diffusive transport roughly corresponds to the average distance between the two target molecules in the sample solution, which can be as large as ˜10 μm at lower concentrations (˜pM). Diffusive transport at that length scale is relatively slow and inefficient, therefore leading to analyte depletion near the binding surface. This can significantly limit the speed of assays, requiring long incubation times to reach binding equilibrium.

Shortening this distance, by using a nanofluidic channel, thereby confining both target molecules and the antibodies is one means pursued, however, the reactions were nonetheless largely diffusion-limited.

### SUMMARY OF THE INVENTION

The invention provides, in one embodiment, a binding assay device, said device comprising:
at least two channels or reservoirs;
at least one nanochannel or nanopores or nanomembrane joining said at least two channels or reservoirs;
a unit through which an electrokinetic or pressure driven flow is induced in said nanochannel; and
optionally, at least one conduit, through which a liquid can be made to pass, linked to said channels;
wherein said nanochannel or nanopore length, the nanochannel height or nanopore diameter, and the local flow velocity in said device are such, that a target molecule or its cognate binding partner introduced in said device has a diffusion time toward a nanochannel or nanopore boundary, which is equal to or larger than a convection time of said target molecule or its cognate binding partner and wherein surfaces of said nanochannel or said nanopore are coated with a material, which is end-functionalized to react selectively with said target molecule.

In one embodiment, the nanochannels are fabricated such that they directly contact the reservoirs, or in another embodiment, the reservoirs are operationally connected to the channels, or in another embodiment, the device comprises only channels and nanochannels. In some embodiments, channels connected to the nanochannels are microchannels.

In one embodiment, the material comprises poly(L-lysine)-g-poly(ethylene glycol), or in another embodiment, other suitable polymers. In one embodiment, the particles are coated with two or more layers of the material, or in another embodiment, the particles are coated with a single layer of said material.

In one embodiment, this invention provides a binding assay device, said device comprising:
at least two channels or reservoirs;
at least one one nanochannel or nanopore or nanomembrane joining said at least two channels or reservoirs, wherein said nanochannel or nanopore or nanomembrane comprises particles coated with a a material, which is end-functionalized to react selectively with a target molecule having a cognate binding partner;
a unit through which an electrokinetic or pressure driven flow is induced in said nanochannel; and
optionally at least one conduit, through which a liquid can be made to pass, linked to said microchannels;
wherein said nanochannel or nanopore length, the nanochannel height or nanopore diameter, and the local flow velocity in said device are such, that a target molecule or its cognate binding partner introduced in said device has a diffusion time toward a nanochannel or nanopore boundary, which is equal to or larger than a convection time of said target molecule or its cognate binding partner and wherein a juncture between said nanochannel and said microchannel prevents particle egress from said nanochannel, and fluid flows freely through said nanochannel.

In one embodiment, the material comprises poly(L-lysine)-g-poly(ethylene glycol), or in another embodiment, other suitable polymers. In one embodiment, the particles are coated with two or more layers of the material, or in another embodiment, the particles are coated with a single layer of said material.

In one embodiment, the material is conjugated to said target molecule, which in one embodiment comprises an antibody, antigen, enzyme, substrate, receptor, ligand, nucleic acid or peptide.

In one embodiment, the target molecule, cognate binding partner, or combination thereof comprises a fluorescent compound.

In one embodiment, the unit through which electrokinetic flow is induced in said nanochannel is connected to a voltage supply, and in another embodiment, the voltage applied by said voltage supply induces an electrokinetic flow. In one embodiment, a pressure driven flow at a velocity ranging from about 1 μm/s-10 m/s is induced in the nanochannel. In some embodiments, exceptionally high flow speeds may be utilized in the devices of this invention, for example, a flow speed of 10 m/s.

In one embodiment, the width of said microchannel is between about 1-1000 μm and the height of the microchannel is between about 0.1-1000 μm. In another embodiment, the characteristic dimension of said nanochannel is between about 1-700 nm.

In another embodiment, the dimensions of nanochannel/nanopore length d, the nanochannel height (pore size) h, and the local flow velocity v are determined in such a way that the diffusion time of the target molecule, cognate binding partner, analyte, etc., toward the wall (˜((h/2)² )/(2 D)) is larger or comparable to the target molecule, or in some embodiments, cognate binding partner, or in some embodiments, analyte, convection time (˜d/v) in the nanochannel/pore.

In another embodiment, the flow velocity (v) is maximized or optimized to allow faster binding and more accurate assays at lower analyte concentrations.

In another embodiment, the devices/methods/kits of this invention provide for increased specificity of binding between target molecules and cognate binding partner.

In another embodiment the devices/methods/kits of this invention provide for the efficient processing of chemicals/molecules, by inducing fast flow through a nanochannel/nanopore/nanomembrane of the devices/kits of the invention, while the enzymes or reactants are immobilized on a surface or wall of the nanochannel/nanopore/nanomembrane.

In another embodiment, the device is comprised of a solid material, which in some embodiments, is Pyrex, silicon dioxide, silicon nitride, silicon, quartz, PDMS or SU-8.

In one embodiment, the device is coupled to an impedance or current meter, or in another embodiment, the device is coupled to a fluorimeter.

In one embodiment, this invention provides an analyte detector, or in another embodiment, a biosensor comprising a device of this invention.

In another embodiment, this invention provides a method for the detection of the binding of a target molecule to a cognate binding partner, the method comprising the steps of:
a. introducing a first liquid comprising a target molecule from a source into a device of this invention, wherein said target molecule specifically interacts with said end-functionalized material on surfaces of said nanochannel;
b. applying a second liquid comprising a cognate binding partner of said target molecule to said device, wherein said second liquid is applied under flow; and
c. measuring changes in a detectable parameter in said device in step (b) versus step (a);
whereby said changes in said detectable parameter indicate said target molecule has bound to a cognate binding partner.

In some embodiments, the second liquid may comprise a mixture of different molecules, and according to this aspect, only a cognate, specific, binding partner interacts with the target molecule, resulting in a detectable/ measurable change.

In one embodiment, this invention provides a method for the detection of the binding of a target molecule to a cognate binding partner, the method comprising the steps of:
a. introducing a first liquid comprising a target molecule from a source into a device of this invention, wherein said target molecule specifically interacts with said end-functionalized material;
b. applying a second liquid comprising a cognate binding partner of said target molecule to the device, wherein said second liquid is applied under flow; and
c. measuring changes in a detectable parameter in the device in step (b) versus step (a);
whereby changes in the detectable parameter indicate said target molecule has bound to a cognate binding partner.

In some embodiments, the detectable parameter is a change in potential, such as, for example, what may be sensed by a FET within the nanochannel. In some embodiments, the detectable parameter is a change in color due to enzyme-substrate reaction, or optical density, or changes in electrochemical activity, for example as measured by amperometric or voltammetric methods.

In one embodiment, the flow is electroosmotic, and in another embodiment, generated by the applied voltage to said device. In one embodiment, the flow is pressure driven and in another embodiment, the pressure driven flow is at a velocity ranging from about 1 μm/s-10 m/s. In another embodiment, the flow is optimized to maximize the speed at which said changes in (c) are detected and minimize disruption of said target molecule binding to a cognate binding partner.

In one embodiment, the first or second liquid is a solution. In another embodiment, the first or second liquid is a suspension, which in another embodiment is an organ homogenate, cell extract or blood sample.

In one embodiment, the target molecule or the binding partner comprises an antibody, antigen, enzyme, substrate, receptor, ligand, nucleic acid or peptide. In another embodiment, the target molecule, said cognate binding partner, or combination thereof comprises a fluorescent compound.

In one embodiment, the method is a screen to identify putative cognate binding partners for said target molecule. In one embodiment, the target molecule is a nucleic acid specifically hybridizing to a molecule comprising a sequence of interest, and said second liquid comprises nucleic acid molecules isolated from a biological sample.

In another embodiment the method is utilized to detect said species of interest when said species is present in said liquid are at low concentration. This method can also be used as a quantitative tool. By measuring the time needed to reach a particular response, the concentration of the analyte can be deduced.

In one embodiment, the method is a diagnostic method. In one embodiment, the method is used to identify biological or environmental toxins in a liquid sample.

In another embodiment, this invention provides a kit for detection of the binding of a target molecule with a cognate binding partner, said kit comprising:
a microfluidic device, said device comprising
   at least two channels or reservoirs;
   at least one nanochannel, nanopore or nanomembrane joining said at least two channels or reservoirs,
   a unit through which an electrokinetic or pressure driven flow is induced in said nanochannel;
   optionally at least one conduit, through which a liquid can be made to pass, linked to said microchannels; and
a material, which is end-functionalized to react selectively with a target molecule having a cognate binding partner; and optionally
a target molecule or a cognate binding partner of interest.

In one embodiment, the kit comprises particles coated with said the material, and in some embodiments, the coating is a mono- or multi-layered coating. In some embodiments, the surface of the nanochannel, nanopore or nanomembrane comprises a mono- or multiplayer of the material. In some embodiments, the particles comprise nanoparticles or quantum dots having electrical or fluorescence properties. In one embodiment, the nanochannel comprises a surface coated with said material. In one embodiment, the material comprises a polymer, such as, for example, poly(L-lysine)-g-poly(ethylene glycol). In one embodiment, the material is conjugated to the target molecule. In one embodiment, the target molecule comprises an antibody, antigen, enzyme, substrate, receptor, ligand, nucleic acid or peptide. In one embodiment, the target molecule, cognate binding partner, or combination thereof comprises a fluorescent compound.

### BRIEF DESCRIPTION OF THE DRAWINGS

FIG. 1 depicts an embodiment of a device of this invention, comprising two microchannels (1-130) joined by nanochannels (1-140). (a) Photograph of the 12×25 mm chip showing the two microchannels and access ports (1-10-1-120). The cross-sectional view along the dotted line is presented in (b), a scanning electron microscope image showing two microchannels with electrodes positioned at their base or bottom, which are connected by nanochannels with height h=50 nm and length d=5.5 μm.

FIG. 2 schematically depicts sequential surface modification of the nanochannels in the devices of this invention, the principle of which is utilized for sensing binding events. (a) depicts a 50-nm-high nanochannel prior to surface modification. (b) depicts the channel following coating with PLL-g-PEG/PEGbiotin, with the monolayer having a height of ˜12 nm. (c) depicts the channel following a streptavidin-biotin reaction, producing a conductance change, which serves as a sensor for the binding reaction.

FIG. 3 plots depicts the difference in conductance in the channel prior to and following streptavidin binding, divided by the conductance prior to binding of 10 μM streptavidin, as a function of the number of nanochannels in the device. The signal change was found to be relatively independent on the number of nanochannels present. For the control measurement the channels were coated with PLL-g-PEG (no biotin), whereas all other chips were pretreated with PLL-g-PEGbiotin.

FIG. 4 plots the normalized conductance change of the nanochannels as a function of streptavidin concentration. After a one hour incubation, ˜0.4 μM streptavidin binding can be detected. The connecting lines are for guidance only.

FIG. 5 plots the differences in conductance over time as a function of flow delivery rate of 1 nM streptavidin. Detection time decreased from ˜12 h to ˜1 h by increasing pressure-driven flow velocity through the nanochannel. The connecting lines are for guidance only.

FIG. 6 plots conductance changes as a function of buffer flow time at a pressure-driven flow rate of ˜3.1 mm/s versus 0 mm/s. Nanochannels pre-coated with PLL-g-PEGbiotin then incubated with 10 μM streptavidin exhibited a signal decrease then achieved equilibrium after ˜2 h, at the higher flow rate, indicating streptavidin-biotin interactions do not readily withstand high shear forces.

FIG. 7 plots the reaction kinetics in nanochannels with an induced flow, measured by the normalized conductance change versus analyte flow time when different streptavidin concentrations are utilized, and flow rate is held constant (˜3.1 mm/s). Dissimilar to standard incubation experiments, in nanochannels the saturation signal changes with the analyte concentration and is reached after equal times of ˜2 h. The connecting lines are for guidance only.

### DETAILED DESCRIPTION OF THE INVENTION

This invention provides, in some embodiments, rapid analyte detection and/or sensor devices/kits and methods of use thereof in the identification of a binding event. Such methods find application in inter alia, immunoassays, screening assays, enzymatic assays, diagnostic assays, screening assays, assays for the identification of biological and/or environmental toxins, and others, as will be appreciated by one skilled in the art.

In some embodiments, the devices/methods/kits of this invention overcome diffusion-limited binding reactions in nanochannels, by applying a convective flow through the channels to enhance binding to a target molecule by enhancing mass transport of its cognate binding pair. Such rapid transport, in turn may allow for fast reaction kinetics in nanofluidic channels and thus a reduction in the response time to detect a specific analyte even at low analyte concentrations, for example, in applications where binding events are the desired readout.

The devices/methods/kits of this invention circumvent surface diffusion layers, in some embodiments, by making use of a device comprising a high ratio of nanochannel length to height, such that target molecules conveyed to the nanochannel will bind to the functionalized surfaces of the channel during their translocation even at high flow velocities.

In some embodiments, the methods/devices/kits of this invention allow for detection of a binding event at a readout/response time decreased by a factor of approximately 54 times that of devices relying on diffusional transport alone, through the described application of flow through the devices/in the methods/kits of this invention.

In some embodiments, the enhanced kinetics as described herein are a function of the application of flow to a device comprising a nanochannel with specific geometry to promote binding to a functionalized surface of the channel.

The invention provides, in one embodiment, a binding assay device, said device comprising:
at least two channels or reservoirs;
at least one nanochannel or nanopores or nanomembrane joining said at least two channels or reservoirs;
a unit through which an electrokinetic or pressure driven flow is induced in said nanochannel; and
optionally, at least one conduit, through which a liquid can be made to pass, linked to said channels; wherein said nanochannel or nanopore length, the nanochannel height or nanopore diameter, and the local flow velocity in said device are such, that a target molecule or its cognate binding partner introduced in said device has a diffusion time toward a nanochannel or nanopore boundary, which is equal to or larger than a convection time of said target molecule or its cognate binding partner and wherein surfaces of said nanochannel or said nanopore are coated with a material, which is end-functionalized to react selectively with said target molecule.

In some embodiments, opposing surfaces of the nanochannel, nanopore or nanomembrane are coated with the material.

In some embodiments, the surfaces are coated with two or more layers of the material, or in some embodiments, the surfaces are coated with a single layer of the material.

In one embodiment, this invention provides a binding assay device, said device comprising:
at least two channels or reservoirs;
at least one one nanochannel or nanopore or nanomembrane joining said at least two channels or reservoirs, wherein said nanochannel or nanopore or nanomembrane comprises particles coated with a a material, which is end-functionalized to react selectively with a target molecule having a cognate binding partner;
a unit through which an electrokinetic or pressure driven flow is induced in said nanochannel; and
optionally at least one conduit, through which a liquid can be made to pass, linked to said microchannels;
wherein said nanochannel or nanopore length, the nanochannel height or nanopore diameter, and the local flow velocity in said device are such, that a target molecule or its cognate binding partner introduced in said device has a diffusion time toward a nanochannel or nanopore boundary, which is equal to or larger than a convection time of said target molecule or its cognate binding partner and wherein a juncture between said nanochannel and said microchannel prevents particle egress from said nanochannel, and fluid flows freely through said nanochannel.

In some embodiments, the devices/methods/kits of this invention allow for enhanced detection/assay of target molecule binding with a cognate partner, which in some embodiments comprises an antibody, nucleic acid, for example, RNA, biomolecules, or other targets of interest. In some embodiments, the application of flow to the devices as described herein allow for the enhancement of binding kinetics through convection, which may be viewed as unexpected, in that the application of flow, which potentially leads to shear stress preventing such interaction, nonetheless facilitated such interaction.

In some embodiments, the dimensions of nanochannel/nanopore length d, the nanochannel height (pore size) h, and the local flow velocity v are determined in such a way that the diffusion time of analyte toward the wall (˜((h/2)² )/(2 D)) is larger or comparable to the analyte convection time (˜d/v) in the nanochannel/pore.

In some embodiments, the thickness of nanochannel/nanopore allows one to meet the said criteria even with very high local flow velocity, potentially up to ˜10 m/s. In such embodiment, analytes or target molecules are conveyed into the nanochannel and readily interact with their cognate binding partner, or vice versa, even at very high local flow speed.

In some embodiments, the flow velocity (v) can be maximized or optimized to allow faster binding and more accurate assays at lower analyte concentrations.

In some embodiments, the enhanced kinetics may be a function of the specific nanoscale reaction volume employed and diffusive transport of a cognate binding pair to bound target molecule, the efficiency of which may be attributable, in part, in some embodiments, to the small length scale of such devices/conditions.

In some embodiments, the fast flow within the said nanochannel/nanopore can be used for eliminating/reducing/mitigating background signals caused by non-specific binding of biomolecules, caused by flow-driven unbinding of non-specific binders. Also, in some embodiments, this mechanism can be used for enhancing specificity of the binding between similar but different molecules, for analysis.

In some embodiments, the diffusion time of molecules in a nanochannel is significantly shorter than their convection time, allowing the molecules a highly enhanced ability to interact/react with molecules present at a surface of the nanochannel/nanopore/nanomembrane, after which such molecules may exit by convection (for example, unbound sample). In some embodiments, this property is used to process chemicals/molecules in the sample liquid while allowing fast flow through the nanochannel/nanopore/nanomembranes.

In some embodiments, the invention provides a binding assay device, said device comprising:
at least two microchannels or reservoirs;
at least one nanochannel joining said at least two microchannels or reservoirs;
a unit through which an electrokinetic or pressure driven flow is induced in said nanochannel; and
optionally at least one conduit, through which a liquid can be made to pass, linked to said microchannels; wherein surfaces of said nanochannel are capable of reacting selectively with a target molecule having a cognate binding partner.

In some embodiments, the term “capable of reacting” refers to treatment of the surface such that selective binding to the target molecule may occur, such as, for example, charging the surface, or applying a compound to the surface which is end-functionalized, but minimally otherwise reacts with e.g., proteins, or other molecules which may interfere with binding to the target molecule.

In some embodiments, the invention provides a binding assay device, said device comprising:
at least two microchannels or reservoirs;
at least one nanochannel joining said at least two microchannels or reservoirs, comprising particles end-functionalized to react selectively with a target molecule having a cognate binding partner;
a unit through which an electrokinetic or pressure driven flow is induced in said nanochannel; and
optionally at least one conduit, through which a liquid can be made to pass, linked to said microchannels;
   wherein a juncture between said nanochannel and said microchannel prevents particle egress from said nanochannel, and fluid flows freely through said nanochannel.

In some embodiments, the particles comprise a material which allows for localized selective surface modifications such that binding of the target molecule is specific. Such functionalized beads are known in the art and readily available from commercial vendors, as will be appreciated by one skilled in the art. In some embodiments, the surfaces of the nanochannel, microchannels, channels or reservoirs as described herein may be selectively modified to facilitate binding, or in some embodiments, to prevent binding, or in some embodiments, facilitate binding in some regions of the devices/kits described herein, and minimize binding in others, or in some embodiments, utilize materials to stimulate binding of certain molecules as a function of the localization within the device.

The devices of this invention may be fabricated via microfabrication technology, or microtechnology or MEMS, in one embodiment, applying the tools and processes of semiconductor fabrication to the formation of, for example, physical structures. Microfabrication technology allows one, in one embodiment, to precisely design features (e.g., wells, channels) with dimensions in the range of<1 mm to several centimeters on chips made, in other embodiments, of silicon, glass, or plastics. Such technology may be used to construct the microchannels of the concentrator, in one embodiment.[the device can be also be made from polymers (e.g. PDMS) using soft lithography/micromolding techniques][the nanochannels could also be formed from materials such as nafion, porous membranes, gels, biological pores, or self assembly of beads].

In another embodiment, NEMS or nanotechnology is used to construct the nanochannels of the devices described herein. In one embodiment, the nanochannels can be fabricated with nanoimprint lithography (NIL), as described in Z. N. Yu, P. Deshpande, W. Wu, J. Wang and S. Y. Chou, Appl. Phys. Lett. 77 (7), 927 (2000); S. Y. Chou, P. R. Krauss, and P. J. Renstrom, Appl. Phys. Lett. 67 (21), 3114 (1995); Stephen Y. Chou, Peter R. Krauss and Preston J. Renstrom, Science 272, 85 (1996) and U.S. Pat. No. 5,772,905 hereby incorporated herein, in their entirety, by reference. In one embodiment, the nanochannels and/or microchannels can be formed by nanoimprint lithography, interference lithography, self-assembled copolymer pattern transfer, spin coating, electron beam lithography, focused ion beam milling, photolithography, reactive ion-etching, wet-etching, plasma-enhanced chemical vapor deposition, electron beam evaporation, sputter deposition, and combinations thereof. Alternatively, other conventional methods can be used to form the nanochannels and/or microchannels.

In one embodiment, the nanochannels and microchannels are formed as exemplified herein below, and as described in J. Han, H. G. Craighead, J. Vac. Sci. Technol., A 17, 2142-2147 (1999) and J. Han, H. G. Craighead, Science 288, 1026-1029 (2000), hereby incorporated fully herein by reference.

In one embodiment, a series of reactive ion etchings are conducted, after which nanochannels are patterned with standard lithography tools. In one embodiment, the etchings are conducted with a particular geometry, which, in another embodiment, determines the interface between the microchannels, and/or nanochannels. In one embodiment, etchings, which create the microchannels, are performed parallel to the plane in which etchings for the nanochannels were created. In another embodiment, additional etching, such as, for example, and in one embodiment, KOH etching was used, to produce additional structures in the concentrator, such as, for example, for creating loading holes.

In another embodiment, electrical insulation of the devices of this invention is conducted. In one embodiment, such insulation is accomplished via nitride stripping and thermal oxidation. In another embodiment, a surface of the device, which in another embodiment is the bottom surface, may be affixed to a substrate, such as, for example, and in one embodiment, a Pyrex wafer. In one embodiment, the wafer may be affixed using anodic bonding techniques or in some embodiments, by a fusion bonding technique.

In one embodiment, construction of the devices of this invention may be accomplished by methods known to one skilled in the art, or adaptation of such methods, such as, for example those described in U.S. Pat. No. 6,753,200, fully incorporated herein by reference.

In one embodiment, the fabrication may use a shaped sacrificial layer, which is sandwiched between permanent floor and ceiling layers, with the shape of the sacrificial layer defining a working gap. When the sacrificial layer is removed, the working gap becomes a fluid channel having the desired configuration. This approach, in one embodiment, allows a precise definition of the height, width and shape of interior working spaces, or fluid channels, in the structure of a fluidic device.

The sacrificial layer is formed on a substrate, is shaped by a suitable lithographic process, for example, and is covered by a ceiling layer. Thereafter, the sacrificial layer may be removed, in some embodiments, with a wet chemical etch or in some embodiments, with a dry isotropic etch, leaving behind empty spaces between the floor and ceiling layers which form working gaps which may be used as flow channels and chambers for the concentrator. In such a device, the vertical dimension, or height, of a working gap is determined by the thickness of the sacrificial layer film, which is made with precise chemical vapor deposition (CVD) techniques, and accordingly, this dimension can be very small.

In order to provide access to the sacrificial layer contained in the structure for the etching solution, which is used to remove the sacrificial layer, one or more access holes may be cut through the ceiling layer, with the wet etch removing the sacrificial layer through these holes. An extremely high etch selectivity may be required between the sacrificial layer and the dielectric layers in order to allow the etch to proceed in the sacrificial layer a significant distance laterally from the access holes without consuming the floor and ceiling layers which compose the finished device. One combination of materials, which may be used for such a process, is polysilicon and silicon nitride, for the sacrificial layer and for the floor and ceiling layers, respectively. Extremely high etch selectivities can be obtained with basic solutions such as, in some embodiments, potassium hydroxide (KOH), sodium hydroxide (NaOH), or in another embodiment, tetramethyl ammonium hydroxide (TMAH).

The access holes cut in the top layer may be covered, in another embodiment. For this purpose, a sealing layer of silicon dioxide may be deposited on top of the ceiling lay to fill in the access holes, and this additional thin film layer provides a good seal against leakage or evaporation of fluids in the working gap. SiO2 CVD techniques, represent other embodiments, which yield a low degree of film conformality, such as very low temperature oxide (VLTO) deposition, form a reliable seal without excessive loss of device area due to clogging near the access holes. If desired, the access holes may be drilled through the bottom layer, instead of or in addition to the holes in the ceiling layer, and later resealed by depositing a layer of silicon dioxide.

For example, in some embodiments, chemical vapor deposition (CVD) may be used to deposit the device materials, including permanent wall materials, which are usually a dielectric material such as silicon nitride or silicon dioxide, and nonpermanent sacrificial layer materials, such as amorphous silicon or polysilicon.

In one embodiment, the microchannels and nanochannel are oriented perpendicularly, with respect to each other. In one embodiment, the term “perpendicular” or “perpendicularly” refers to an orientation of one channel being at a 90° angle with respect to the longitudinal axis of another channel, +/−5 or in another embodiment, at a 90° angle of +/−10°, or in another embodiment, at a 90° angle +/−20°. In another embodiment, the microchannels and nanochannel are oriented such that the long axis of each channel is oriented along the same Cartesian axis, for example, the long axis of the microchannels and the long axis of the nanochannel are oriented horizontally, in parallel to the long axis of a device of this invention, with the nanochannel flanked by the two microchannels.

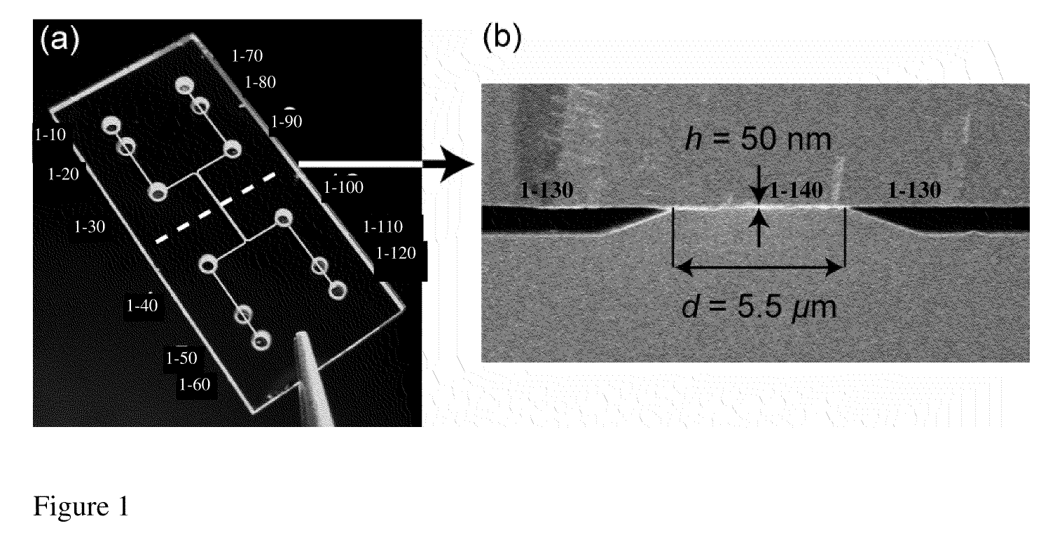

In one embodiment, FIG. 1 presents one envisioned device of this invention, where a series of access ports provide for fluidic access and waste, and electrical contact can be made through pads in other access ports. Multiple chip configurations can be envisioned, for example as described herein, where 1, 2, 5, and 10 nanochannels join two microchannels. Each nanochannel, in this embodiment of the device has a height of 50 nm, a width of 50 μm, and length of 5.5 μm, thus the width is several orders of magnitude larger than the height, which in some embodiments, promotes analyte detection, or in other embodiments, facilitates greater diffusive transport, or a combination thereof, in the channel.

In one embodiment, an interface region is constructed which connects the microchannels and nanochannel of the concentrator of this invention. In one embodiment, diffraction gradient lithography (DGL) is used to form a gradient interface between the microchannels and nanochannels of this invention. In one embodiment, the gradient interface region may regulate flow through the devices of this invention, or in another embodiment, trap particles in the nanochannels and prevent their egress to adjacent microchannels.

In one embodiment, the gradient interface area is formed of lateral spatial gradient structures for narrowing the cross section of a value from the micron to the nanometer length scale. In another embodiment, the gradient interface area is formed of a vertical sloped gradient structure. In another embodiment, the gradient structure can provide both a lateral and vertical gradient.

In one embodiment, the devices of this invention may be fabricated by diffraction gradient lithography, by forming a nanochannel or nanochannels on a substrate, forming a microchannel or microchannels on the substrate and forming a gradient interface area between them. The gradient interface area can be formed, in one embodiment, by using a blocking mask positioned above a photo mask and/or photoresist during photolithography. The edge of the blocking mask provides diffraction to cast a gradient light intensity on the photoresist.

In one embodiment, the devices of this invention may comprise a plurality of channels, including a plurality of microchannels, or a plurality of nanochannels, or a combination thereof. In one embodiment, the phrase “a plurality of channels” refers to any desired number of channels, which may be accommodated in the devices of this invention, and the skilled artisan will appreciate the construction of such devices to suit a desired purpose.

In one embodiment, the width of the microchannel is between 1-1000 μm, or in another embodiment, between 1 and 150 μm, or in another embodiment, between 20 and 500 μm, or in another embodiment, between 25 and 750 μm, or in another embodiment, between 50 and 1000 μm. In one embodiment, the depth/height of the microchannel is between 0.1-1000 μm, or in another embodiment, between 0.1 and 500 μm, or in another embodiment, between 5 and 150 μm, or in another embodiment, between 10 and 250 μm, or in another embodiment, between 15 and 500 μm.

In one embodiment, the height of the microchannel is between 1-1000 nm, or in another embodiment, between 100 and 500 nm, or in another embodiment, between 250 and 750 nm, or in another embodiment, between 500 and 100 nm, or in another embodiment, 850 nm.

In another embodiment, the width of the nanochannel is between 10 nm-1000 μm, or in another embodiment, between 10 and 750 μm, or in another embodiment, between 25 and 500 μm, or in another embodiment, between 15 and 400 μm, or in another embodiment, between 50 and 1000 μm.

In another embodiment, the height of said nanochannel is between 2-700 nanometers, or in another embodiment, between 2 and 50 nanometers, or in another embodiment, between 2 and 75 nanometers, or in another embodiment, between 35 and 75 nanometers, or in another embodiment, between 2 and 20 nanometers, or in another embodiment, about 50 nanometers.

In another embodiment, the length of said nanochannel is between 0.1-1000 μm, or in another embodiment, between 0.1 and 15 μm, or in another embodiment, between 0.1 and 10 μm, or in another embodiment, between 0.5-10 μm, or in another embodiment, about 5 μm.

In some embodiments, the reservoirs and channels as described herein are several micron in size, in any dimension, or in some embodiments, such structures are macroscopic in scale, for example, from a few millimeters in size to a few centimeters. In some embodiments, the reservoirs have no size restriction.

In some embodiments, the devices may comprise a large number of parallel nanopores/nanochannels on a large membrane (for example, a 6 inch wafer) connecting two reservoirs (for example tens of centimeters in size), which may find application in many assay systems, for example, such as in chemical processing, where for example, enzymes are immobilized at a surface wall of a nanopore.

In some embodiments, the dimensions of the device are such that flow is optimized by adjusting the nanochannel length versus height in consideration of the diffusivity of the compound. In some embodiments, the length and height of the nanochannel can be independently and arbitrarily controlled. In some embodiments, construction is such that the diffusion time of a target molecule introduced into the device is comparable to or less than its convection time (((h/2)2 )/(2 D)≦d/v). In some embodiments, velocity is maximally increased, yet not resulting in so high a flow speed that interaction of the target molecule and its cognate binding partner is disrupted.

In some embodiments, the dimensions of the device are such that, considering the diffusion equation t=(x²)/(2 D), the design criterion should consider: ((h/2)²)/(2 D)≦d/v.

In one embodiment, the device of this invention is constructed as diagrammed in FIG. 1.

In one embodiment, the flow induced in the device is nonlinear electroosmotic flow generated in the microchannel, which draws fluid into the microchannels from the sample reservoir with high flow speed, and because an energy barrier for anionic molecules is generated by the induced space charge layer in the microchannel, at regions of apposition to the nanochannels.

In one embodiment, the flow may be pressure-driven, and may be accomplished by any means well known to one skilled in the art. In another embodiment, the flow may be a hybrid of pressure-driven and electrokinetic flow.

In one embodiment, the phrases “pressure-driven flow” refers to flow that is driven by a pressure source external to the channel segment through which such flow is driven, as contrasted to flow that is generated through the channel segment in question by the application of an electric field through that channel segment, which is referred to herein, in one embodiment, as “electrokinetically driven flow.”

Examples of pressure sources include negative and positive pressure sources or pumps external to the channel segment in question, including electrokinetic pressure pumps, e.g., pumps that generate pressure by electrokinetically driven flow in a pumping channel that is separate from the channel segment in question, provided such pumps are external to the channel segment in question (see, U.S. Pat. Nos. 6,012,902 and 6,171,067, each of which is incorporated herein by reference in its entirety for all purposes).

In one embodiment, a pressure driven flow at a velocity ranging from about 0.1 μm/s-10 m/s is induced in the nanochannel.

In one embodiment, the term “electrokinetic flow” refers to the movement of fluid or fluid borne material under an applied electric field. Electrokinetic flow generally encompasses one or both of electrophoresis, e.g., the movement of charged species through the medium or fluid in which it is disposed, as well as electroosmosis, e.g., the electrically driven movement of the bulk fluid, including all of its components. Accordingly, when referred to in terms of electrokinetic flow, it will be appreciated that what is envisioned is the full spectrum of electrokinetic flow from predominantly or substantially completely electrophoretic movement of species, to predominantly electroosmotically driven movement of material, e.g., in the case of uncharged material, and all of the ranges and ratios of the two types of electrokinetic movement that fall between these extremes.

In one embodiment, reference to the term “liquid flow” may encompass any or all of the characteristics of flow of fluid or other material through a passage, conduit, channel or across a surface. Such characteristics include without limitation the flow rate, flow volume, the conformation and accompanying dispersion profile of the flowing fluid or other material, as well as other more generalized characteristics of flow, e.g., laminar flow, creeping flow, turbulent flow, etc.

In one embodiment, hybrid flow may comprise pressure-based relay of the liquid sample into the channel network, followed by electrokinetic movement of materials, or in another embodiment, electrokinetic movement of the liquid followed by pressure-driven flow.

In one embodiment, an electric field may be induced in the respective channels by applying voltage from a voltage supply to the device. In one embodiment voltage is applied by way of the placement of at least one pair of electrodes capable of applying an electric field across at least some of the channels in at least one direction. Electrode metal contacts can be integrated using standard integrated circuit fabrication technology to be in contact with at least one microchannel, or in another embodiment, at least one nanochannel, or in another embodiment, a combination thereof, and oriented as such, to establish a directional electric field. Alternating current (AC), direct current (DC), or both types of fields can be applied. The electrodes can be made of almost any metal, and in one embodiment, comprise thin Al/Au metal layers deposited on defined line paths. In one embodiment, at least one end of one electrode is in contact with buffer solution in the reservoir.

In one embodiment, the unit through which electrokinetic flow is induced in the nanochannel is connected to a voltage supply, and in another embodiment, the voltage applied by said voltage supply does induce an electrokinetic flow.

In another embodiment, the devices of this invention may contain at least two pairs of electrodes, each providing an electric field in different directions. In one embodiment, field contacts can be used to independently modulate the direction and amplitudes of the electric fields to, in one embodiment, orient the space charge layer, or in another embodiment, move macromolecules at desired speed or direction, or in another embodiment, a combination thereof.

In one embodiment, the voltage applied does induce an electrokinetic flow.

In one embodiment, the voltage supply may be any electrical source, which may be used to provide the desired voltage. The electrical source may be any source of electricity capable of generating the desired voltage. For example, the electrical source may be a piezoelectrical source, a battery, or a device powered by household current. In one embodiment, a piezoelectrical discharge from a gas igniter may be used.

In one embodiment, the binding of a target molecule to a cognate binding partner in the device can occur over a course of seconds, or in another embodiment, minutes, or in another embodiment, several hours. In one embodiment, binding rate may be optimized by adjusting the conditions employed during such assay, such as by modifying the interface between the microchannel and nanochannel, voltage applied, salt concentration of the liquid, pH of the liquid, temperature or environmental conditions, or a combination thereof.

In another embodiment, the devices of this invention further comprises at least one waste reservoir in fluid communication with the microchannel, microchannels, nanochannel or nanochannels of the devices of this invention. In one embodiment, the waste reservoir is capable of receiving a fluid.

In one embodiment, the surface of the microchannel may be functionalized to enhance adsorption of the non-conductive material to the surface of the devices of this invention. In another embodiment, the device is comprised of a solid material. In another embodiment, the solid material is Pyrex, silicon dioxide, silicon nitride, silicon, quartz or SU-8 or polymer.

In some embodiments, this invention provides methods/devices/kits, which comprise the application of flow to devices comprising nanogaps, nanochannels, nanopores, nanogels, nanomembranes, or any nanoscale spaces, wherein the geometry is such, that following specific binding of a target molecule to opposing surfaces in said nanogaps, nanochannels, nanopores, nanogels, nanomembranes, or any nanoscale spaces, or to particles trapped within said nanogaps, nanochannels, nanopores, nanogels, nanomembranes, or any nanoscale spaces, and application of a liquid comprising a cognate binding partner under flow to such a device, enhanced binding kinetics will occur, which may be detected, or made use of, representing embodiments of this invention.

In some embodiments, according to this aspect, devices as described in Joon Sung Lee, et al., Mat. Res. Soc. Symp. Proc. Vol. 729 ©2002 Materials Research Society, pages U4.10.1-U4.10.6; or Im H. et al., Nature Nanotechnology (2007) Vol 2: 430-435; U.S. Patent Application Publication No. 2005/0074778, and others, as will be appreciated by one skilled in the art, may be utilized as biosensors, convective mixers, as herein described, wherein such devices are adapted such that a flow may be induced in the nanogaps, nanochannels, nanopores, nanogels, nanomembranes, or any nanoscale spaces described therein, where the dimensions of such nanogaps, nanochannels, nanopores, nanogels, nanomembranes, or any nanoscale spaces, are such so as to promote rebounding of a cognate binding partner off a surface of such structures, or surrounding materials, which create such structures, thereby enhancing binding kinetics as herein described.

In one embodiment, the device is coupled to an impedance or current meter, or in another embodiment, the device is coupled to a fluorimeter. In some embodiments, coupling the device to other machinery used in the analysis of materials contained therein effects the methods as herein described.

In one embodiment, this invention provides an analyte detector, or in another embodiment, a biosensor comprising a device of this invention.

Fast reaction kinetics can be achieved in nanogaps, nanochannels, nanopores, nanogels, nanomembranes, or any nanoscale spaces with a convective flow through them, in some embodiments, because there is no limiting diffusion layer as in standard incubation experiments. Low analyte concentrations may be detected electrically with impedance spectroscopy or current measurements, as exemplified and described herein, taking advantage of the device design and application of flow, as described herein.

Channel surfaces coating with an end-functionalized protein-resistant monolayer, enables widespread applications of the devices as described herein to function as a biosensor.

While high shear forces due to the applied flow result could result in decrease or breakage of the binding events, which are a desired product/readout of the devices/methods/kits of this invention minimizing such shear forces may be accomplished and optimum conditions may be arrived at which result in the shortest response time and greatest detection limit.

Normalized conductance changes between 11-24% were exemplified herein, based on the devices/methods/kits/principles described herein, and enhanced kinetics would be expected in similar systems employing particle-conjugated target molecules, as described herein. According to this aspect, and in one embodiment, the ratio of bead-diameter to nanochannel height would be quite large, whereupon immobilization of beads to the walls the geometrical cross-section of the nanochannel decreases, which can, e.g. be electrically measured at high ionic strength. It is to be understood that the skilled artisan can readily arrive at an optimum between bead-size, detection limit, flow velocity, and response time by for example, evaluating these parameters by methods and under conditions, inter alia, as exemplified herein.

In another embodiment, this invention provides a method for the detection of the binding of a target molecule to a cognate binding partner, the method comprising the steps of:
a. introducing a first liquid comprising a target molecule from a source into a device of this invention, wherein said target molecule specifically interacts with said end-functionalized material;
b. applying a second liquid comprising a cognate binding partner of said target molecule to the device of this invention, wherein said second liquid is applied under flow; and
c. measuring changes in a detectable parameter in said device in step (b) versus step (a);
whereby said changes in said detectable parameter indicate said target molecule has bound to a cognate binding partner.

In another embodiment, this invention provides a method for the detection of the binding of a target molecule to a cognate binding partner, the method comprising the steps of:
a. introducing a first liquid comprising a target molecule from a source into a device of this invention, wherein said target molecule specifically interacts with said end-functionalized material on opposing surfaces of said nanochannel;
b. applying a second liquid comprising a cognate binding partner of said target molecule to said device, wherein said second liquid is applied under flow; and
c. measuring changes in conductance, capacitance, color, optical density, potential, electrochemical activity or fluorescence in said device in step (b) versus step (a);
whereby said changes in conductance, capacitance, color, optical density, potential, electrochemical activity or fluorescence indicate said target molecule has bound to a cognate binding partner.

In one embodiment, this invention provides a method for the detection of the binding of a target molecule to a cognate binding partner, the method comprising the steps of:
a. introducing a first liquid comprising a target molecule from a source into a device of this invention, wherein said target molecule specifically interacts with said end-functionalized material on said particles;
b. applying a second liquid comprising a cognate binding partner of said target molecule to said device, wherein said second liquid is applied under flow; and
c. measuring changes in changes in conductance, capacitance, color, optical density, potential, electrochemical activity or fluorescence in said device in step (b) versus step (a);
   whereby said changes in conductance, capacitance, color, optical density, potential, electrochemical activity or fluorescence indicate said target molecule has bound to a cognate binding partner.

In some embodiments, changes in conductance, capacitance or fluorescence can be conducted as described and exemplified herein. Such devices and apparatuses for the determination of conductance, capacitance or fluorescence prior to and following the binding events described are well known to the skilled artisan, for example, using an LCR or impedance meter, a current meter, a fluorimeter, and others as will be appreciated by one skilled in the art.

In some embodiments, the measured change is in conductance, and detection is as described and exemplified herein. In some embodiments, according to this aspect, binding of the target molecule and cognate binding partner in the nanochannels, result in conductance increases associated with an increase in the surface charge due to the additional charge of e.g. the binding partner, with such conductance increases serving as a readout or indicator for a binding event in this aspect of the invention. In some embodiments, the devices/methods/kits/principles described herein provide a significantly more rapid readout for a binding event for nanodevices known to date. In some embodiments, when solutions/suspensions at high ionic concentration are utilized in the devices/kits/methods of this invention, a conductance decrease may be associated with a binding event due to blockage of the nanochannel, which in turn may also serve as a readout/indicator for a binding event, and comprises a method of this invention.

In some embodiments, the devices/methods/kits/principles of this invention make use of fluorescently labeled target molecule, which is adhered to opposing surfaces of a device, or in some embodiments, to surfaces of beads as described herein, localized within nanochannels, etc. of devices as described herein. According to this aspect, and in one embodiment, the cognate binding partner comprises a fluorescent compound as well, and the rapid conveyance of the partner molecule and subsequent binding event results in fluorescent resonance energy transfer (FRET), which may be readily ascertained, as is well known in the art, for example, through the use of a fluorimeter.

In another embodiment, the devices of this invention are adapted such that analysis of a species of interest may be conducted, in one embodiment, in the devices of this invention, or in another embodiment, downstream of the devices of this invention. In one embodiment, analysis downstream of the concentrator refers to removal of the target molecule or in another embodiment, removal of the cognate binding partner from the device, and placement in an appropriate setting for analysis, or in another embodiment, construction of a conduit from the devices of this invention which relays the target molecule or cognate binding partner to an appropriate setting for analysis. In one embodiment, such analysis may comprise signal acquisition, and in another embodiment, a data processor. In one embodiment, the signal can be a photon, electrical current/impedance measurement or change in measurements. It is to be understood that the devices of this invention may be useful in various analytical systems, including bioanalysis Microsystems, due to its simplicity, performance, robustness, and integrabilty to other separation and detection systems, and any integration of the device into such a system is to be considered as part of this invention.

In another embodiment the devices of this invention or in another embodiment, the nanochannel or nanochannels are capable of being imaged with a two-dimensional detector. Imaging of the devices of this invention, or parts thereof, may be accomplished by presenting it to a suitable apparatus for the collection of emitted signals, such as, in some embodiments, optical elements for the collection of light from the nanochannels.

In another embodiment, the device is coupled to a separation system, or in another embodiment, a detection system, or in another embodiment, an analysis system or in another embodiment, a combination thereof. In another embodiment, the device is coupled to an illumination source.

In one embodiment, the devices of this invention may be disposable, and in another embodiment, may be individually packaged, and in another embodiment, have a sample loading capacity of 1-50,000 individual fluid samples. In one embodiment, the devices of this invention can be encased in a suitable housing, such as plastic, to provide a convenient and commercially-ready cartridge or cassette. In one embodiment, the devices of this invention will have suitable features on or in the housing for inserting, guiding, and aligning the device, such that, for example, a sample loading compartment is aligned with a reservoir in another device, which is to be coupled to the devices of this invention. For example, the devices of this invention may be equipped with insertion slots, tracks, or a combination thereof, or other adaptations for automation of the methods/applications of devices/kits of this invention.

The devices of this invention may be so adapted, in one embodiment, for high throughput screening of multiple samples, such as will be useful in genomics or proteomics applications, as will be appreciated by one skilled in the art.

In one embodiment, the devices of this invention are connected to electrodes, which are connected to an electric potential generator, which may, in another embodiment be connected with metal contacts. Suitable metal contacts can be external contact patches that can be connected to an external scanning/imaging/electric-field tuner, in another embodiment.

In one embodiment of the present invention, the devices of this invention are a part of a larger system, which includes an apparatus to excite molecules inside the channels and detect and collect the resulting signals. In one embodiment, a laser beam may be focused upon the target molecule or bound cognate partner, using a focusing lens, in another embodiment. The generated light signal from the molecules inside the nanochannels may be collected by focusing/collection lens, and, in another embodiment, reflected off a dichroic mirroriband pass filter into optical path, which may, in another embodiment, be fed into a CCD (charge coupled device) camera.

In another embodiment, an exciting light source could be passed through a dichroic mirror/band pass filter box and focusing/collecting scheme from the top of the devices of this invention. Various optical components and devices can also be used in the system to detect optical signals, such as digital cameras, PMTs (photomultiplier tubes), and APDs (Avalanche photodiodes).

In another embodiment, the system may further include a data processor. In one embodiment, the data processor can be used to process the signals from a CCD, to a digital image of the concentrated species onto a display. In one embodiment, the data processor can also analyze the digital image to provide characterization information, such as size statistics, histograms, karyotypes, mapping, diagnostics information and display the information in suitable form for data readout.

In one embodiment, the target material or cognate binding partner comprises an active agent, which allows for conductance of assays in the nanochannel, whose efficiency may in some embodiments, be a reflection of changes in conductance, capacitance, field effects, fluorescence, etc., as will be appreciated by the skilled artisan.

For example, and in one embodiment, the target material comprises an enzyme and the cognate binding partner comprises a substrate with which the enzyme interacts, and kinetics of the reaction there-between are such that changes in conductance, capacitance, field effects, fluorescence, etc., indicative of the proximity of the two parallels reaction completion, for example wherein the rate limiting step of such reactions is the proximal localization of the enzyme and substrate.

In some embodiments of this invention, the enzyme is a protease, and the invention provides a method for proteome analysis, wherein, for example, a sample comprising a plurality of cellular polypeptides is concentrated in the microchannel, to obtain a plurality of substantially purified polypeptides. The polypeptide is exposed to a protease bound to a non-conductive material within the nanochannel (e.g. coated on opposing surfaces of the channel or adhered to beads immobilized within the channel), under conditions sufficient to substantially digest the polypeptide, thereby producing digestion products or peptides. The digestion products may, in another embodiment, then be transported to a downstream separation module where they are separated, and in another embodiment, from there, the separated digestion products may be conveyed to a peptide analysis module. The amino acid sequences of the digestion products may be determined and assembled to generate a sequence of the polypeptide. Prior to delivery to a peptide analysis module, the peptide may be conveyed to an interfacing module, which in turn, may perform one or more additional steps of separating, concentrating, and or focusing.

In other embodiments, the proteases include, but are not limited to: peptidases, such as aminopeptidases, carboxypeptidases, and endopeptidases (e.g., trypsin, chymotrypsin, thermolysin, endoproteinase Lys C, endoproteinase GluC, endoproteinase ArgC, endoproteinase AspN). Aminopeptidases and carboxypeptidases are useful in characterizing post-translational modifications and processing events. Combinations of proteases also can be used.

In one embodiment, the proteases and/or other enzymes are localized within the nanochannel by adsorption or covalent bonding to the channel surface or particle surface, as described herein. In some embodiments, the protease is attached to such surfaces or particles which have been coated with a non-conductive material which is end-functionalized.

In other embodiments, the target molecule/binding partner pairs may include the following: cytostatin/papain, valphosphanate/carboxypeptidase A, biotin/streptavidin, riboflavin/riboflavin binding protein, antigen/antibody binding pairs, receptor/ligand, protein/protein (e.g. multiple proteins in a signaling cascade), protein/DNA, DNA/RNA, DNA/cDNA, or others as will be appreciated by the skilled artisan.

In one embodiment, the steps of assaying polypeptides obtained from a given cell, producing digestion products, and analyzing digestion products to determine protein sequence, can be performed in parallel and/or iteratively for a given sample.

In one embodiment, the first or second liquid applied as described in the methods of this invention is a solution. In some embodiments, the solution comprises the target molecule or in another embodiment, the cognate binding partner. In some embodiments, both the target molecule and the cognate binding partner are soluble in solution. In some embodiments, the solution characteristics facilitate rapid binding and assay of the binding event.

In another embodiment, the first or second liquid is a suspension, which in another embodiment is an organ homogenate, cell extract or blood sample. In some embodiments, digestion of a biological sample in a buffer is performed, prior to application of a suspension of the digested products to the device, as described herein. In some embodiments, such digested products may be further processed and/or purified, prior to their application, for example, via subjection to differential centrifugation.

In some embodiments, solutions or suspensions of biological materials are utilized in the devices/methods/kits of this invention, and materials of interest therein may comprise the target molecule or binding partner source. In some embodiments, such use will find application in diagnostics and other screening methods as will be appreciated by one skilled in the art.

In one embodiment, the target molecule or cognate binding pair comprises an antibody, antigen, enzyme, substrate, receptor, ligand, nucleic acid or peptide.

In some embodiments, the term “target molecule” is any molecule with which another specifically interacts, wherein the interaction is of interest, and may be determined using the devices or methods or kits of this invention. In some embodiments, the term “cognate binding partner” refers to a second molecule, which specifically interacts with the target molecule. In some embodiments, the “target molecule” and “cognate binding partner” comprise a binding pair. In some embodiments, multiple target molecules have the same cognate binding partner, and in some embodiments, multiple cognate binding partners bind the same target molecule.

In one embodiment, the method is a screen to identify putative cognate binding partners for said target molecule. In one embodiment, the target molecule is a nucleic acid specifically hybridizing to a molecule comprising a sequence of interest, and said second liquid comprises nucleic acid molecules isolated from a biological sample.

In another embodiment the method is utilized to detect said species of interest when said species is present in said liquid at a concentration which is below a limit of detection.

In one embodiment, the method is a diagnostic method. In one embodiment, the method is used to identify biological or environmental toxins in a liquid sample.

In one embodiment, this invention provides an array architecture that is capable of being scaled to be suitable for a real-world screen.

In one embodiment, the methods of this invention may be conducted under controlled physicochemical parameters, which may comprise temperature, pH, salt concentration, or a combination thereof.

In one embodiment, the method further comprises the step of releasing the target molecule or cognate binding partner, or combination thereof from the device. In one embodiment, the method further comprises the step of subjecting the target molecule or cognate binding partner, or combination thereof to capillary electrophoresis.

Capillary electrophoresis is a technique that utilizes the electrophoretic nature of molecules and/or the electroosmotic flow of samples in small capillary tubes to separate sample components. Typically a fused silica capillary of 100 μm inner diameter or less is filled with a buffer solution containing an electrolyte. Each end of the capillary is placed in a separate fluidic reservoir containing a buffer electrolyte. A potential voltage is placed in one of the buffer reservoirs and a second potential voltage is placed in the other buffer reservoir. Positively and negatively charged species will migrate in opposite directions through the capillary under the influence of the electric field established by the two potential voltages applied to the buffer reservoirs. The electroosmotic flow and the electrophoretic mobility of each component of a fluid will determine the overall migration for each fluidic component. The fluid flow profile resulting from electroosmotic flow is flat due to the reduction in frictional drag along the walls of the separation channel. The observed mobility is the sum of the electroosmotic and electrophoretic mobilities, and the observed velocity is the sum of the electroosmotic and electrophoretic velocities.

In one embodiment of the invention, a capillary electrophoresis system is micromachined onto a device, which is a part of, or separate from the devices of this invention. Methods of micromachining capillary electrophoresis systems onto devices are well known in the art and are described, for example in U.S. Pat. No. 6,274,089; U.S. Pat. No. 6,271,021; Effenhauser et al., 1993, Anal. Chem. 65: 2637-2642; Harrison et al., 1993, Science 261: 895-897; Jacobson et al., 1994, Anal. Chem. 66:1107-1113; and Jacobson et al., 1994, Anal. Chem. 66: 1114-1118.

In one embodiment, the capillary electrophoresis separations provide a sample which may then be used for both MALDI-MS and/or ESI-MS/MS-based protein analyses (see, e.g., Feng et al., 2000, Journal of the American Society For Mass Spectrometry 11: 94-99; Koziel, New Orleans, La. 2000; Khandurina et al., 1999, Analytical Chemistry 71: 1815-1819. Such separations, for example, may find application in the screening methods as described herein, for the identification of fished cognate binding partners, whose interaction with the target molecule was heretofore unknown.

In other embodiments, upstream or downstream separation devices, which may interface with the devices of this invention include, but are not limited to, micro high performance liquid chromatographic columns, for example, reverse-phase, ion-exchange, and affinity columns.

It is to be understood that the exact configuration of any systems, devices, etc. which are coupled upstream or downstream of the devices of this invention are to be considered as part of this invention, and that the configuration may be varied, to suit a desired application.

In some embodiments, the devices of this invention are useful as biosensor devices. In one embodiment, such devices/methods/kits are particularly useful in detecting organisms in a latent or spore state, wherein detection of the organism is otherwise difficult.

In other embodiments, various applications of the methods of the present invention are possible without deviating from the present invention.

By way of example, the devices/methods/kits of the present invention allow for high-throughput robotic assaying systems, to screen for a species of interest, or a binding partner of interest, and other applications, which may be applicable, inter alia, in screening promising drug candidates derived from libraries, for example, whose binding to a particular target molecule is of interest, or in some embodiments, in screening for the identification of molecular targets for drug design, for example screening for the identification of proteins, which interact with viral or bacterial cytotoxins, and others as will be appreciated by the skilled artisan.

In another embodiment, this invention provides a kit for detection of the binding of a target molecule with a cognate binding partner, said kit comprising:
a microfluidic device, said device comprising
   at least two channels or reservoirs;
   at least one nanochannel joining said at least two channels or reservoirs,
   a unit through which an electrokinetic or pressure driven flow is induced in said nanochannel;
   optionally at least one conduit, through which a liquid can be made to pass, linked to said microchannels; and
a material, which is end-functionalized to react selectively with a target molecule having a cognate binding partner; and
optionally a target molecule or a cognate binding partner of interest.

In another embodiment, this invention provides a kit for detection of the binding of a target molecule with a cognate binding partner, said kit comprising:
a microfluidic device, said device comprising
   at least two microchannels or reservoirs;
   at least one nanochannel joining said at least two microchannels or reservoirs,
   a unit through which an electrokinetic or pressure driven flow is induced in said nanochannel; and
   optionally at least one conduit, through which a liquid can be made to pass, linked to said microchannels; and
a material, which is end-functionalized to react selectively with a target molecule having a cognate binding partner; and optionally
a target molecule or a cognate binding partner of interest.

In one embodiment, the kit comprises surfaces or particles coated with the material, which may be present as a mono- or multiplayer coating. In one embodiment, the nanochannel comprises particles coated with the material. In one embodiment, the material comprises any suitable polymer, for example poly(L-lysine)-g-poly(ethylene glycol). In one embodiment, the material is conjugated to the target molecule. In one embodiment, the target molecule comprises an antibody, antigen, enzyme, substrate, receptor, ligand, nucleic acid or peptide. In one embodiment, the target molecule, cognate binding partner, or combination thereof comprises a fluorescent compound.

Various modes of carrying out the invention are contemplated as being within the scope of the following claims particularly pointing out and distinctly claiming the subject matter, which is regarded as the invention.

### EXAMPLES

### Materials and Methods

### Device Fabrication:

Nanochannels were fabricated in Pyrex using fusion bonding for their encapsulation. Two microchannels were bulk-micromachined in Pyrex, and under-etching was reduced by means of a polysilicon mask. Then, 100-nm-thick platinum electrodes with a 10-nm-thin adhesion layer of titanium were patterned at the bottom of the microchannels using a standard lift-off technique. The two microchannels were connected by etching the nanochannels. Buffered oxide etch (7:1), used in nanochannel etching, allowed precise channel depth control since it has an etch-rate of 24 nm/min in glass at room temperature [see Mao, P.; Han, J. Lab Chip 2005, 5, 837, incorporated herein by reference in its entirety].

Glass-glass fusion bonding of the wafer containing the micro- and nanochannels to the Pyrex wafer was accomplished with ultrasonically pre-drilled holes (SENSOR Prep Services, Inc., Elburn, Ill.), both wafers were cleaned with a Piranha process followed by surface activation in a heated ammonium hydroxide bath for 30 min. Thereafter, both wafers were assembled to form a spontaneous bonding between them, and they were subsequently annealed at 550° C. overnight. Afterwards, the wafers were diced into chips such that they could be placed into a chip holder with integrated o-rings and spring-loaded contacts, allowing convenient fluidic and electrical connections.

### Conductance Measurements

The conductance of the nanochannels was measured with impedance spectroscopy, performed with the precision LCR meter E4980A (Agilent Technologies, Inc., Englewood, Colo.) in the range of 20 Hz-2 MHz with a peak-to-peak voltage of 50 mV. The instrument was controlled by a Matlab or LabView interface program, and signal was measured as described previously [Schoch, R. B.; van Lintel, H.; Renaud, P. Phys. Fluids 2005, 17, 100604.1].

### Binding Assay

Nanochannel surfaces were pre-coated with the commercially available polymer PLL(20)-g[3.5]-PEG(2)/PEG(3.4)-Biotin (50%) (SurfaceSolutionS, Zurich, Switzerland) at 0.1 mg/ml, referred to herein as “PLL-g-PEGbiotin”. Control measurements were performed by modifying surfaces with a layer which is highly effective in reducing the adsorption of proteins [Pasche, S. et al. Langmuir 2003, 19, 9216, fully incorporated by reference herein], PLL(20)-g[3.5]-PEG(2) (SurfaceSolutionS, Zurich, Switzerland) at 0.1 mg/ml, referred to herein as “PLL-g-PEG”. A 10 mM HEPES buffer solution (Sigma-Aldrich, St. Louis, Mo.), adjusted to pH 7.4 with NaOH (Sigma-Aldrich, St. Louis Mo.), which has an equivalent ionic strength of ˜5.6 mM and a Debye length λ_{D}=4.1 nm was utilized unless otherwise specified. Chips were stocked in the buffer solution for at least 48 h before use.

A vacuum was applied to device waste reservoirs, the chip was flushed with buffer solution for 10 minutes. PLL-g-PEG/PEGbiotin was applied to the channels, which were incubated for 1 hour, to form a monolayer coating. Chips were rinsed 30 minutes with buffer solution to remove excess polymer. PLL-g-PEG/PEGbiotin was desorbed from the electrode surfaces by applying 1.8 V between the microfabricated electrodes and the reservoirs for 30 minutes, which did not induce polymer loss from the silicon oxide regions [Tang, C. S. et al., J. Biotechnol. Bioeng. 2005, 91, 285]. Subsequently, the channels were rinsed twice with buffer solution, for 30 min and 1 h.

Micro- and nanochannels were filled with a streptavidin solution (at various concentrations), incubated for 1 hour without any induced flow or subjected to a continuous fluid flow through the nanochannels. Such fluid flow was either pressure driven (syringe pump PHD 2000 Infuse/Withdraw (Harvard Apparatus, Holliston, Mass.), and the magnitude has been verified with particle image velocimetry measurements in situ, near the nanochannels,) or electro-osmotically driven (by using an electric field of 160 V/cm). Channels were then rinsed with buffer solution for 30 minutes and the conductance of the nanochannel was measured, as described. Between experiments, channels were cleaned for 1 hour with 1 M sodium chloride and 1 wt % SDS, to remove PLL-g-PEG/PEGbiotin, verified by measuring conductance before and after cleaning.

### Example 1

### Rapid Convection and Sensing Device

FIG. 1 describes an embodiment of a device of this invention, depicting a chip comprising nanochannels (1-140), placed proximally to abut microchannels (1-130). Access ports 1-10-1-60 are positioned to the left of the long axis of the microchannels depicted, and holes 1-70-1-120 to the right of the long axis of the microchannels depicted.

Ports 1-30/1-90 and 1-40/1-100 provide fluidic access to the microchannel, e.g. for introduction of sample and waste removal of each microchannel, respectively. Electrical contact can be made through the pads in ports 1-10/1-70 or 1-60/1-120, respectively. To control the pressure in the chip, and/or prevent liquid flow into the electrical contact sites, ports 1-20/1-50 and 1-80/1-110 may be sealed with nonconductive glue.

Different chip configurations were fabricated with 1-10, 1-20, 1-50, and 1-100 nanochannels joining the two microchannels. Each nanochannel had the following dimensions: height h=50 nm, width w=50 μm, and length d=5.5 μm. The microchannels are 850 nm high and 50 μm wide.

The embodied device was then evaluated in terms of conductance of the nanochannels, as measured with impedance spectroscopy in the range of 20Hz-2 MHz with a peak-to-peak voltage of 50 mV. The instrument was controlled by a Matlab or LabView interface program. The measured signal at a given frequency, which is ˜500 Hz for the investigated nanochannels and electrolyte solutions, corresponded to the resistance of the nanochannel junction between the two microchannels, as a function of electrode placement closely to both ends of the nanochannels, such that the resistance of the nanochannel would be dominant over other resistive components like the microchannel solution resistance, for example.

The arrangement of the nanochannels between microchannels, as described in the embodied device, for example, differs from other such devices, nanogaps are used to detect biomolecules through changes in the dielectric constant of the gap, using impedance measurements over the height of the nanogap.

### Example 2

### Detection of a Binding Event in Embodied Devices of this Invention

For electrical detection of immobilized proteins in nanochannels, streptavidin-biotin was chosen as the model receptor-ligand pair. To perform such bindings in nanochannels, surfaces were pre-coated with PLL(20)-g[3.5]-PEG(2)/PEG(3.4)-Biotin (50%) at 0.1 mg/ml (hereinafter referred to as “PLL-g-PEGbiotin”). This polymer is end-functionalized with biotin and therefore reacts selectively with streptavidin. Controls included surfaces pre-coated with 0.1 mg/ml PLL(20)-g[3.5]-PEG(2) (hereinafter referred to as “PLL-g-PEG”) layer, which reduces protein adsorption. The polymers are known to spontaneously adsorb from aqueous solutions to oxide surfaces due to the positively charged poly(L-lysine) group at neutral pH, are protein-resistant due to the poly(ethylene glycol) group forming a comblike structure, and can be end-functionalized to react selectively with a target molecule.

10 mM HEPES buffer solution having an equivalent ionic strength of ˜5.6 mM and a Debye length λ_{D}=4.1 nm was used. At this ionic strength the PLL-g-PEG/PEGbiotin layer is sufficiently thick to shield electrical double layer forces since the monolayer thickness of ˜12 nm exceeds the Debye length [Pasche, S. et al., M. J. Phys. Chem. B 2005, 109, 17545].

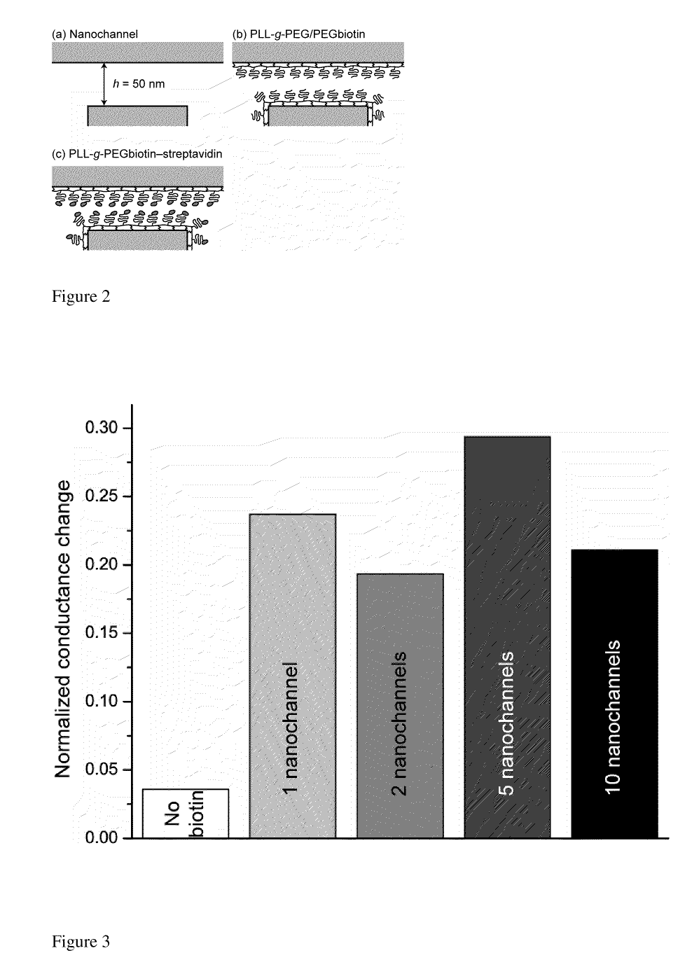

PLL-g-PEG/PEGbiotin formed a monolayer on the nanochannel, as depicted in FIG. 2(b). To ensure that the polymer adsorbed on the electrodes does not change the electrical signal, PLL-g-PEG/PEGbiotin was desorbed from the electrode surfaces specifically, polymer loss from silicon oxide regions did not occur.

Micro- and nanochannels were then filled with a streptavidin solution at various concentrations. The solutions were applied without any induced flow (streptavidin binding was diffusion limited), or a continuous fluid flow was applied (streptavidin binding was convectively driven) [FIG. 2(c)].

Since streptavidin has an individual molecular size of about 5 nm, the final polymer layer (after streptavidin binding to PEGbiotin) would decrease the effective nanochannel height to ˜16 nm. The channels were rinsed and assessed for conductance.

Multiple embodiments of devices/chips of the invention were prepared, comprising 1, 2, 5, and 10 nanochannels. Such embodiments were then filled as described above, and binding of streptavidin at a high concentration (10 μM) was determined. The results are presented in FIG. 3. The streptavidin concentration and incubation time ensured that saturation occurred, resulting in maximal binding to the nanochannel wall.

Normalized conductance reflects the difference between the conductance before and after streptavidin binding, divided by the conductance before binding, and a positive value therefore reflects a conductance increase.

Normalized conductance varied between 19% and 29%, which is attributed to differences in the surface charge density of the nanochannels but not the number of nanochannels. This is due to variations in the native surface charge of SiO₂, leading to differences in monolayer and hence streptavidin densities. The surface of these channels was modified with PLL-g-PEGbiotin except for the control measurement in which nanochannels were coated with a protein resistant PLL-g-PEG monolayer, leading to a normalized conductance change of 3.6%. This value is slightly higher than the repeatability error limit of ˜3%, confirming a negligible amount of nonspecific protein adsorption.

Following channel coating with PLL-g-PEG/PEGbiotin, normalized conductance of the nanochannels decreased by ˜50%, corroborating that the nanochannel height h decreased from 50 nm to ˜25 nm due to the ˜12 nm thick polymer monolayers. PEG coating is uncharged, therefore leading to neutral nanochannels whose conductance is entirely described by the geometry of the nanometer-sized openings.

The binding reaction between PLL-g-PEGbiotin and streptavidin would reduce the effective height of the nanochannel down to ˜16 nm. Since streptavidin has a net charge of about −2 e at pH 7.4 [Sivasankar, S. et al. PNAS 1998, 95, 12961], the nanochannel surface is more negatively charged post-binding, with a corresponding higher conductance, since conductance is dominated by surface charge density.

Using the model of Schoch, R. B. and Renaud, P. (Appl. Phys. Lett. 2005, 86, 253111.1), when a maximal surface coverage γ₀=2.4×10¹⁶ of streptavidin molecules/m² [Jung, L. S. et al. Langmuir 2000, 16, 9421] is present, a normalized conductance change of ˜15% is calculated, close to the measured average value of 24% in the figure.

### Example 3

### Concentration-Dependent Effects on Diffusive Binding

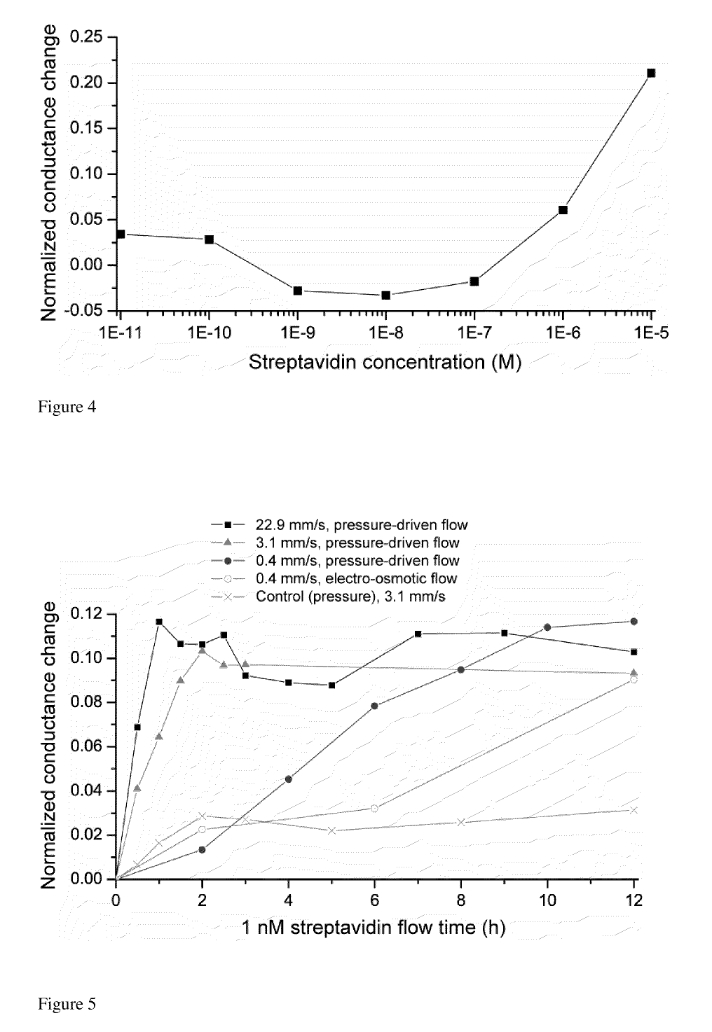

To determine the lowest detectable concentration of biomolecules in a diffusion-limited reaction, the normalized conductance change was investigated as a function of the streptavidin concentration (FIG. 4). In this aspect, the lowest detectable streptavidin concentration in nanochannels is estimated to be 0.4 μM. At lower biomolecule concentrations, detected nanochannel conductance changes were within the repeatability error of ˜3%. The poor detectability at lower streptavidin concentrations can be attributed, in part, to the failure to achieve binding equilibrium.

The failure of detection of the lowest streptavidin concentrations in FIG. 4 may be attributable to the process of diffusion-limited patterning of nanochannels [see Karnik et al. Karnik, R.; Castelino, K.; Duan, C.; Majumdar, A. Nano Lett. 2006, 6, 1735]. Under diffusion, the coating time tdiff of a nanochannel with a length d is:

$${t}_{diff} = \frac{P {γ}_{0} {d}^{2}}{2 DAc} \qquad ( 1 )$$

where P is the perimeter of the nanochannel cross-section, D is the diffusion constant of the analyte (6×10⁻¹¹ m²/s for streptavidin), A is the cross-section of the nanochannel, and c is the streptavidin concentration. According to eq (1), the coating time is proportional to d², and inversely proportional to the analyte concentration c. This time is calculated to be as long as t_{diff}≈54 h for 1 nM streptavidin solution in the nanochannels of the exemplified embodiments of devices of this invention, assuming γ₀=2.4×10¹⁶ m⁻² as described above.

Another parameter influencing streptavidin interaction with its binding partner is the time required for streptavidin traversal of the microchannel (where the compound is introduced) toward the nanochannel inlet (transport at the micro-nanochannel interface). This transition from microchannel to nanochannel can be affected by the formation of an analyte depletion zone near the nanochannel inlet, as well as steric hindrance of biomolecules at the micro-nanochannel junction. Delays attributable thereto, however, are expected to be negligible.

### Example 4

### Flow Effects on Binding

The response time was significantly improved over diffusion-mediated binding, when pressure-driven or electro-osmotic flow was applied through the nanochannels.

Flux is characterized by φ=Avc, where v is the velocity of the liquid, and when in quasi-steady state, the rate of consumption of the nanochannel is Pγ₀(dx/dt), which is equal to the flux of streptavidin [Kamik, R. et al., Nano Lett. 2006, 6, 1735]. The coating time under flow is calculated to be:

$${t}_{flow} = \frac{P {γ}_{0} d}{Avc} \qquad ( 2 )$$

Under active flow the response time is now proportional to d, rather than d², as seen in the diffusion-limited binding regime. Moreover, t_{flow} can be further reduced by increasing the flow velocity v through the nanochannel. The only limitation to this mode of reaction kinetics enhancement would arise when the limit of the average analyte transit time through the nanochannel (d/v) is comparable to the vertical diffusion time within the nanochannel ((h/2)²/2 D). In other words, analytes will pass the nanochannel without ever diffusing to the surface of the nanochannel in this limit.

According to the embodied device and conditions utilized in the Examples herein, this limit corresponds to vₜ≈3.9 m/s, largely due to the small height h of the nanochannel.

Conductance measurements indicate reduced response time, depending upon the flow type and velocity applied (FIG. 5). For a pressure-driven flow velocity of ˜0.4 mm/s, maximal normalized conductance changes are only obtained after ˜12 h, and this time is reduced to ˜2 h by imposing v≈3.1 mm/s. Further increasing this velocity to ˜22.9 mm/s reduces the response time to ˜1 h. Flow velocity increased as a function of time, in some embodiments, as a function of pressure build-up in the Tygon® tubing used.

The highest generated flow velocity of ˜22.9 mm/s was limited by the maximal force of the syringe pump, but velocities up to vₜ≈3.9 m/s could theoretically be used as estimated above by equating time scales of imposed axial flow and radial diffusional transport.

Electro-osmotic flow was also utilized as a mean to induce flow through the nanochannel. A flow rate of ˜0.4 mm/s under an electric field of 160 V/cm was utilized, and this voltage did not lead to bubble generation.

A control of 1 nM streptavidin solution alone applied at a flow velocity of 3.1 mm/s to PLL-g-PEG coated channels, did not lead to streptavidin binding yet reduced nonspecific protein adsorption almost completely.

The calculated response time for 0.4 mm/s is 11.5 h (eq 2), which approximately corresponds to the measured 12 h. Theoretical response times for 3.1 mm/s and 22.9 mm/s were predicted to be 1.5 h and 0.2 h, respectively, which were shorter than the measured values of ˜2 h and ˜1 h.

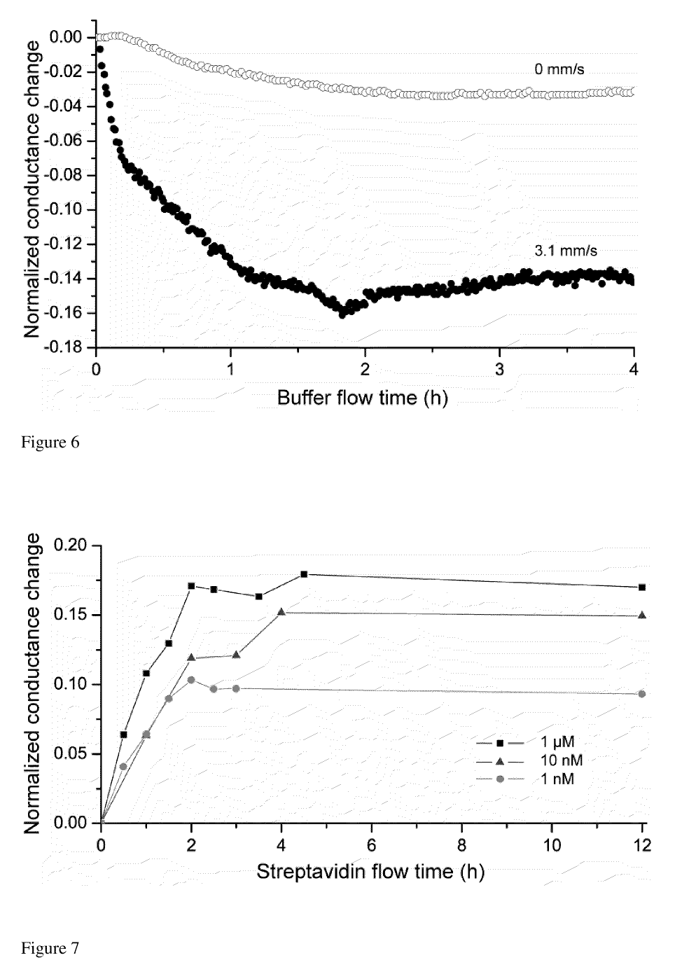

To understand these differences, a reference experiment was performed in which nanochannels were coated with PLL-g-PEGbiotin and incubated in a 10 μM streptavidin solution for 1 h. Subsequently, the nanochannels were flushed with buffer solution only at a flow velocity of ˜3.1 mm/s (and zero flow velocity for control) as shown in FIG. 6. It has been observed that the normalized conductance change decreased to ˜14% after ˜2 h, reaching equilibrium. This conductance decrease is associated with a reduced number of streptavidin molecules in the nanochannels, because high forces could lead to a dissection of the streptavidin-biotin bond despite its dissociation constant of 4×10⁻¹⁴ M. Force-induced breaking of streptavidin-biotin binding has been measured on the pN level. Based on the hydrodynamic drag force given by Stoke's law, bond-breaking is possible since the shear force acting on the target molecule is in the pN range at this flow velocity and size of the streptavidin molecule (approximately 5 nm in diameter). Since PLL-g-PEG has also been shown to be useful as a lubrication layer in devices subjected to up to velocities of some m/s, a detachment of the entire polymer from the surface is unlikely.

The results of FIGS. 5 and 6 indicate the ability to arrive at an optimum flow velocity that results in a short response time, without inducing bond cleavage between binding partners. This optimum flow velocity can be calculated by Stoke's law as described above for a force below rupture of the receptor-ligand interaction.

FIG. 6 indicates that target molecules can also be detected if bond association and dissociation occurs at high flow velocity, although at a reduced conductance signal, which allows a decrease in the response time, a finding with important applications for immunoassays.

The saturation of the normalized conductance change after ˜2 h, seen in FIG. 6, may be due to analyte replenishment. Another advantage of high flow velocities could be reduced weak and non-specific binding which break at high shear forces.

In diffusion-limited binding assays, the same saturation signal is obtained for all analyte concentrations, but after different times. Fundamentally different reaction kinetics result in nanochannels with an induced flow as shown in FIG. 7, where the streptavidin flow time as a function of the normalized conductance change is presented for different streptavidin concentrations at a flow velocity of ˜3.1 mm/s. In nanofluidic channels the saturation signal changes with the analyte concentration, and saturations are observed after ˜2 h for all streptavidin concentrations.

In this aspect, the saturation signal represents an equilibrium between streptavidin-biotin bond association and dissociation, with the change in the saturation value being due, in some embodiments, to analyte replenishment which decreases with dilution. This determines the detection limit of this nanochannel-flow biosensor, because only analyte concentrations above the repeatability error can be measured. Since streptavidin-biotin bond breakage only leads to a constant conductance change after ˜2 h at v≈3.1 mm/s (FIG. 6), this process does limit the response time, leading to about equal times to reach saturation for different analyte concentrations.

Some embodiments of this invention are directed to the fast reaction kinetics achieved in nanochannels with a convective flow through them, because there is no limiting diffusion layer as in standard incubation experiments.

Some embodiments of this invention are directed to electric low analyte concentration detection with impedance spectroscopy, when channel surfaces are coated with an end-functionalized protein-resistant monolayer, which in turn may find application in sensor technology, for example, biosensor technology.

In some embodiments of the invention, conductance change values can be increased by using conjugated beads to bind to binding partner-coated nanochannel surfaces, and in some embodiments, the ratio of bead-diameter to nanochannel height is large. Upon immobilization of beads to the walls, the geometrical cross-section of the nanochannel will decrease, which can be electrically measured at high ionic strength. In some embodiments, bead size, flow velocity and response time desired can be optimized, such that shear stress effects on the material/binding partner are minimized, as will be appreciated by one skilled in the art.

In some embodiments, the devices and methods as described herein are particularly usefully applied to immunoassays, and other assays relying on a material binding to a binding partner, such as enzyme substrate interaction, antigen-antibody interation, DNA-protein interaction, and others.

While certain features of the invention have been illustrated and described herein, many modifications, substitutions, changes, and equivalents will now occur to those of ordinary skill in the art. It is, therefore, to be understood that the claims are intended to cover all such modifications and changes as fall within the true spirit of the invention.

What is claimed is:

1. A binding assay device, said device comprising:
at least two channels or reservoirs;
at least one nanochannel or nanopores or nanomembrane joining said at least two channels or reservoirs;
a unit through which an electrokinetic or pressure driven flow is induced in said nanochannel; and
optionally, at least one conduit, through which a liquid can be made to pass, linked to said channels;
wherein said nanochannel or nanopore length, nanochannel height or nanopore diameter, and local flow velocity in said device are such, that a target molecule or its cognate binding partner introduced in said device has a diffusion time toward a nanochannel or nanopore boundary, which is equal to or larger than a convection time of said target molecule or its cognate binding partner and wherein surfaces of said nanochannel or said nanopore are coated with a material, which is end-functionalized to react selectively with said target molecule.

2. The device of claim 1, wherein said surfaces are coated with two or more layers of said material.

3. The device of claim 1, wherein said surfaces are coated with a single layer of said material.

4. The device of claim 1, wherein said material comprises poly(L-lysine)-g-poly(ethylene glycol).

5. The device of claim 1, wherein said material is conjugated to said target molecule.

6. The device of claim 1, wherein said target molecule and/or is binding partner on the surface comprises an antibody, antigen, enzyme, substrate, receptor, ligand, nucleic acid or peptide.

7. The device of claim 1, wherein said target molecule, said cognate binding partner, or combination thereof comprises a fluorescent compound.

8. The device of claim 1, wherein said means for inducing electrokinetic flow in said nanochannel is a voltage supply.

9. The device of claim 8, wherein said voltage applied by said voltage supply induces an electrokinetic flow.

10. The device of claim 1, wherein said pressure driven flow is at a velocity ranging from about 1 μm/s-10 m/s.

11. The device of claim 1, wherein said channel is a microchannel.

12. The device of claim 11, wherein the width of said microchannel is between about 1-1000 μm and the height of the microchannel is between about 0.1-1000μm.

13. The device of claim 1, wherein the width of said nanochannel is between about 10 nm-1000 μm, the length of the nanochannel is between about 0.1-1000 μm, and the height of the nanochannel is between about 1-700 nm.

14. The device of claim 1, wherein said device is comprised of a solid material.

15. The device of claim 11, wherein said solid material is Pyrex, silicon dioxide, silicon nitride, silicon, quartz, SU-8 or polydimethylsiloxane (PDMS).

16. The device of claim 1, wherein said device is coupled to an impedance or current meter.

17. The device of claim 1, wherein said device is coupled to a fluorimeter.

18. The device of claim 1, wherein said device comprises multiple microchannels and nanochannels.

19. A convective analyte detector, comprising the device of claim

20. A biosensor comprising the device of claim 1.

21. A chemical reactor comprising the device of claim 1.

22. A method for the detection of the binding of a target molecule to a cognate binding partner, the method comprising the steps of:
a. introducing a first liquid comprising a target molecule from a source into the device of claim 1, wherein said target molecule specifically interacts with said end-functionalized material on surfaces of said nanochannel;
b. applying a second liquid comprising a cognate binding partner of said target molecule to said device of claim 1, wherein said second liquid is applied under flow; and
c. measuring changes in a detectable parameter in said device in step (b) versus step (a);
whereby said changes in a detectable parameter indicate said target molecule has bound to a cognate binding partner.

23. The method of claim 22, wherein said flow is electroosmotic.

24. The method of claim 23, wherein a voltage is applied to said device to induce an electrokinetic flow.

25. The method of claim 22, wherein said flow is pressure driven.

26. The method of claim 25, wherein said pressure driven flow is at a velocity ranging from about 1 μm/s-10 m/s.

27. The method of claim 25, wherein said flow is optimized to maximize the speed at which said changes in (c) are detected and minimize disruption of said target molecule binding to a cognate binding partner.

28. The method of claim 26, wherein steps are carried out cyclically.

29. The method of claim 22, wherein said first or second liquid is a solution.

30. The method of claim 22, wherein said first or second liquid is a suspension.

31. The method of claim 30, wherein said suspension is an organ homogenate, cell extract or blood sample.

32. The method of claim 22, wherein said target molecule comprises an antibody, antigen, enzyme, substrate, receptor, ligand, nucleic acid or peptide.

33. The method of claim 22, wherein said target molecule, said cognate binding partner, or combination thereof comprises a fluorescent compound.

34. The method of claim 22, wherein said method is a screen to identify putative cognate binding partners for said target molecule.

35. The method of claim 34, wherein said target molecule is a nucleic acid specifically hybridizing to a molecule comprising a sequence of interest, and said second liquid comprises nucleic acid molecules isolated from a biological sample.

36. The method of claim 22, wherein said method is utilized to detect said species of interest when said species is present in said liquid at a low concentration.

37. The method of claim 22, wherein said method is a diagnostic method.

38. The method of claim 22, wherein said method is used to identify biological or environmental toxins in a liquid sample.

39. A binding assay device, said device comprising:
at least two channels or reservoirs;
at least one nanochannel or nanopore or nanomembrane joining said at least two channels or reservoirs, wherein said nanochannel or nanopore or nanomembrane comprises particles coated with a material, which is end-functionalized to react selectively with a target molecule having a cognate binding partner;
a unit through which an electrokinetic or pressure driven flow is induced in said nanochannel; and
optionally at least one conduit, through which a liquid can be made to pass, linked to said microchannels;
wherein said nanochannel or nanopore length, the nanochannel height or nanopore diameter, and the local flow velocity in said device are such, that a target molecule or its cognate binding partner introduced in said device has a diffusion time toward a nanochannel or nanopore boundary, which is equal to or larger than a convection time of said target molecule or its cognate binding partner and wherein a juncture between said nanochannel and said microchannel prevents particle egress from said nanochannel, and fluid flows freely through said nanochannel.

40. The device of claim 39, wherein said material comprises poly(L-lysine)-g-poly(ethylene glycol).

41. The device of claim 39, wherein said particles are coated with two or more layers of said material.

42. The device of claim 39, wherein said particles are coated with a single layer of said material.

43. The device of claim 39, wherein said material is conjugated to said target molecule.

44. The device of claim 39, wherein said target molecule comprises an antibody, antigen, enzyme, substrate, receptor, ligand, nucleic acid or peptide.

45. The device of claim 39, wherein said target molecule, said cognate binding partner, or combination thereof comprises a fluorescent compound.

46. The device of claim 39, wherein said means for inducing electrokinetic flow in said nanochannel is a voltage supply.

47. The device of claim 46, wherein said voltage applied by said voltage supply does induce an electrokinetic flow.

48. The device of claim 39, wherein said pressure driven flow is at a velocity ranging from about 1 μm/s-10 m/s.

49. The device of claim 39, wherein the width of said microchannel is between about 1-1000 μm and the height of the microchannel is between about 0.1-1000 μm.

50. The device of claim 39, wherein the width of said nanochannel is between about 10 nm-1000 μm, the length of the nanochannel is between about 0.1-1000 μm, and the height of the nanochannel is between about 1-700 nm.

51. The device of claim 39, wherein said device is comprised of a solid material.

52. The device of claim 51, wherein said transparent material is Pyrex, silicon dioxide, silicon nitride, silicon, quartz, SU-8, or polydimethylsiloxane (PDMS).

53. The device of claim 39, wherein said device is coupled to an impedance or current meter.

54. The device of claim 39, wherein said device is coupled to a fluorimeter.

55. The device of claim 39, wherein said device comprises multiple microchannels and nanochannels.

56. A convective analyte detector, comprising the device of claim 39.

57. A biosensor comprising the device of claim 39.

58. A method for the detection of the binding of a target molecule to a cognate binding partner, the method comprising the steps of:
a. introducing a first liquid comprising a target molecule from a source into the device of claim 39, wherein said target molecule specifically interacts with said end-functionalized material;
b. applying a second liquid comprising a cognate binding partner of said target molecule to said device of claim 39, wherein said second liquid is applied under flow; and
c. measuring changes in a detectable parameter in said device in step (b) versus step (a);

59. whereby said changes in said detectable parameter indicate said target molecule has bound to a cognate binding partner. The method of claim 58, wherein said parameter is conductance, capacitance, fluorescence, surface potential changes, optical density, electrochemical activity or a combination thereof.

60. The method of claim 58, wherein said flow is electroosmotic.

61. The method of claim 58, wherein a voltage is applied to said device to induce an electrokinetic flow.

62. The method of claim 58, wherein said flow is pressure driven.

63. The method of claim 62, wherein said pressure driven flow is at a velocity ranging from about 1 μm/s-10 m/s.

64. The method of claim 58, wherein said flow is optimized to maximize the speed at which said changes in (c) are detected and minimize disruption of said target molecule binding to a cognate binding partner.

65. The method of claim 58, wherein steps are carried out cyclically.

66. The method of claim 58, wherein said first or second liquid is a solution.

67. The method of claim 58, wherein said first or second liquid is a suspension.

68. The method of claim 67, wherein said suspension is an organ homogenate, cell extract or blood sample.

69. The method of claim 58, wherein said target molecule comprises an antibody, antigen, enzyme, substrate, receptor, ligand, nucleic acid or peptide.

70. The method of claim 58, wherein said target molecule, said cognate binding partner, or combination thereof comprises a fluorescent compound.

71. The method of claim 58, wherein said method is a screen to identify putative cognate binding partners for said target molecule.

72. The method of claim 71, wherein said target molecule is a nucleic acid specifically hybridizing to a molecule comprising a sequence of interest, and said second liquid comprises nucleic acid molecules isolated from a biological sample.

73. The method of claim 58, wherein said method is utilized to detect said species of interest when said species is present in said liquid at a concentration which is below a limit of detection.

74. The method of claim 58, wherein said method is a diagnostic method.

75. The method of claim 58, wherein said method is used to identify biological or environmental toxins in a liquid sample.

76. A kit for detection of the binding of a target molecule with a cognate binding partner, said kit comprising:
a microfluidic device, said device comprising
at least two channels or reservoirs;
at least one nanochannel joining said at least two channels or reservoirs,
a unit through which an electrokinetic or pressure driven flow is induced in said nanochannel;
optionally at least one conduit, through which a liquid can be made to pass, linked to said microchannels; and
a material, which is end-functionalized to react selectively with a target molecule having a cognate binding partner; and optionally
a target molecule or a cognate binding partner of interest.
wherein said nanochannel or nanopore length, nanochannel height or nanopore diameter, and local flow velocity in said device are such, that a target molecule or its cognate binding partner introduced in said device has a diffusion time toward a nanochannel or nanopore boundary, which is equal to or larger than a convection time of said target molecule or its cognate binding partner.

77. The kit of claim 76, wherein said kit comprises particles coated with said material.

78. The kit of claim 77, wherein said device further comprises a juncture between said nanochannel and said channels or said reservoirs, which prevents particle egress from said nanochannel, and fluid flows freely through said nanochannel.

79. The kit of claim 77, wherein said nanochannel comprises particles coated with said material.

80. The method of claim 79, wherein particles are coated with a mono- or multi-layer of said material.

81. The kit of claim 76, wherein surfaces of said channels or reservoirs are coated with said material.

82. The method of claim 81, wherein said material is applied to said surfaces as a mono- or multi-layer.

83. The kit of claim 76, wherein said material comprises poly(L-lysine)-g-poly(ethylene glycol).

84. The kit of claim 76, wherein said material is conjugated to said target molecule.

85. The kit of claim 76, wherein said target molecule comprises an antibody, antigen, enzyme, substrate, receptor, ligand, nucleic acid or peptide.

86. The kit of claim 76, wherein said target molecule, said cognate binding partner, or combination thereof comprises a fluorescent compound.
